(19) United States
(12) Patent Application Publication
(10) Pub. No.: US 2024/0294858 A1
(43) Pub. Date: Sep. 5, 2024

(54) STIRRING DEVICES FOR BIOREACTORS

(71) Applicant: ALIT BIOTECH (SHANGHAI) CO., LTD., Shanghai (CN)

(72) Inventors: Guoqian XU, Shanghai (CN); Pengrong XU, Shanghai (CN); Rui CHEN, Shanghai (CN)

(73) Assignee: ALIT BIOTECH (SHANGHAI) CO., LTD., Shanghai (CN)

(21) Appl. No.: 18/593,905

(22) Filed: Mar. 2, 2024

(30) Foreign Application Priority Data
Mar. 2, 2023 (CN) 202310203119.8

(51) Int. Cl. C12M 1/06; C12M 1/00; C12M 3/00

(52) U.S. Cl. CPC C12M 27/02; C12M 23/46; C12M 23/50

(57) ABSTRACT

The present disclosure provides a stirring device for a bioreactor. The bioreactor includes a tank for accommodating a mixture of cells and liquid. The stirring device includes a rotating shaft, a magnetic member, and a connecting member. A bottom portion of the rotating shaft is connected to the magnetic member. The magnetic member is in no contact with a bottom portion of the tank to drive the rotating shaft. The rotating shaft is in no contact with the bottom portion of the tank. The rotating shaft is connected to a top portion of the tank through the connecting member.

### CROSS-REFERENCE TO RELATED APPLICATION

The present disclosure claims priority to Chinese patent application No. 202310203119.8, filed on Mar. 2, 2023, the entire contents of which are incorporated herein by reference.

### TECHNICAL FIELD

The present disclosure relates to the technical field of bioreactor devices, and in particular, to a stirring device for a bioreactor.

### BACKGROUND

A bioreactor is a device used for cell proliferation, monitoring, and maintaining cell physiological status. Cell tissues that have been genetically modified or edited can be placed in the bioreactor for proliferation. During cell proliferation, substances in the bioreactor need to be mixed and stirred. Currently, a stirring device is usually fixed in the bioreactor with a fixed sleeve and rotates around the fixed sleeve under a traditional mechanical stirrer to carry out stirring operations. However, as usage time increases, wear and tear between the traditional mechanical stirrer and the fixed sleeve can not only shorten the service life and range of the bioreactor but also cause particulate contamination.

Therefore, there is a need to provide a stirring device for a bioreactor that reduces particulate contamination, with a long service life and wide service range.

### SUMMARY

One of the embodiments of the present disclosure provides a stirring device for a bioreactor. The bioreactor may include a tank for accommodating a mixture of cells and liquid. The stirring device may include a rotating shaft, a magnetic member, and a connecting member, a bottom portion of the rotating shaft may be connected to the magnetic member, the magnetic member may be in no contact with a bottom portion of the tank to drive the rotating shaft to rotate; the rotating shaft may be in no contact with the bottom portion of the tank; and the rotating shaft may be connected to a top portion of the tank through the connecting member.

In some embodiments, the connecting member may include a bearing assembly, the bearing assembly may be connected to the tank through a fixing member; the fixing member may be an integral part of the tank, the fixing member may be disposed on a lower surface of the top portion of the tank, and the fixing member may be in no contact with the rotating shaft.

In some embodiments, the connecting member may include a first position-limiting member disposed at the top portion of the tank and a second position-limiting member disposed at a top portion of the rotating shaft, and the first position-limiting member may be connected with the second position-limiting member.

In some embodiments, the stirring device may further include an envelope, the envelope may be disposed around the connecting member, and a lower end of the envelope may be fixedly connected to the rotating shaft with no gap, and an upper end of the envelope may be connected with the connecting member.

In some embodiments, the connecting member may be connected with an inner side of the upper end of the envelope, and the rotating shaft may be inserted through a through-hole at the lower end of the envelope, and an outer peripheral side of the rotating shaft may be connected with the through-hole at the lower end of the envelope with no gap.

In some embodiments, a water-blocking disk may be disposed at a top portion of the envelope, the water-blocking disk may be located above the envelope, and a bottom portion of the water-blocking disk may be connected to the top portion of the envelope.

In some embodiments, there may be no holes on a disk surface of the water-blocking disk.

In some embodiments, a center region of the water-blocking disk may be higher than a rim region of the water-blocking disk.

In some embodiments, a disk surface of the water-blocking disk may be provided with a concentric circular groove, and a surface of the concentric circular groove may be lower than two rim regions of the water-blocking disk.

In some embodiments, a lower surface of a top portion of the tank may include one or more annular convex structures, the one or more annular convex structures being of different diameters and co-circular; an upper surface of the water-blocking disk may include a convex loop, the convex loop may include one or more concentric rings of different diameters; and each of the one or more concentric rings of the convex loop may be embedded in a gap between two adjacent annular convex structures of the one or more annular convex structures.

In some embodiments, the convex loop may be in no contact with the one or more annular convex structures along a radial direction of the tank to form a zigzag channel between the convex loop and the one or more annular convex structures.

In some embodiments, a vacuuming and liquid-collecting member may be provided inside the envelope, the vacuuming and liquid-collecting member being fixed at a bottom region inside the envelope.

In some embodiments, at least one opening may be provided in the vacuuming and liquid-collecting member.

In some embodiments, a separating plate may be disposed at a bottom portion inside the envelope, the separating plate may be configured to separate the vacuuming and liquid-collecting member from a top portion of the rotating shaft; and the vacuuming and liquid-collecting member may be disposed above the separating plate, and the top portion of the rotating shaft may be located below the separating plate.

In some embodiments, the tank may include an enclosure disposed underneath the top portion of the tank, the enclosure may be connected with a lower surface of the top portion of the tank and at least a portion of the enclosure may be disposed around the connecting member, and a height of the enclosure may be greater than a distance between an upper surface of the connecting member and the lower surface of the top portion of the tank.

In some embodiments, the tank may include an enclosure disposed underneath the top portion of the tank, the enclosure may be connected with a lower surface of the top portion of the tank and at least a portion of the enclosure may be disposed around the envelope, and a height of the enclosure may be greater than a distance between an upper surface of the envelope and the lower surface of the top portion of the tank.

In some embodiments, a position-limiting member may be disposed at a bottom portion of the enclosure, and a radial distance between the position-limiting member and the rotating shaft along a radial direction of the tank may be less than a radial width of the connecting member along the radial direction of the tank.

In some embodiments, the position-limiting member may include one or more locking ribs distributed in an array with a center of a circle of the enclosure.

In some embodiments, the fixing member may be connected with the bearing assembly through a telescoping member.

One of the embodiments of the present disclosure provides a bioreactor comprising a tank for accommodating a mixture of cells and liquid and the stirring device as described in any of the preceding embodiments.

### BRIEF DESCRIPTION OF THE DRAWINGS

The present disclosure will be further illustrated by way of exemplary embodiments, which will be described in detail by means of the accompanying drawings. These embodiments are not limiting, and in these embodiments, the same numbering denotes the same structure, wherein:

FIG. 1 is a schematic diagram illustrating an exemplary bioreactor according to some embodiments of the present disclosure;

FIG. 2 is a schematic diagram illustrating an exemplary stirring device according to some embodiments of the present disclosure;

FIG. 3 is a schematic diagram illustrating an exemplary connecting member according to some embodiments of the present disclosure;

FIG. 4A is a schematic diagram illustrating an exemplary connecting member including a bearing assembly that includes a radial bearing according to some embodiments of the present disclosure;

FIG. 4B is a schematic diagram illustrating an exemplary connecting member including a bearing assembly that includes an axial bearing according to some embodiments of the present disclosure;

FIG. 5 is a schematic diagram illustrating an exemplary envelope according to some embodiments of the present disclosure;

FIG. 6A is a schematic diagram illustrating an exemplary water-blocking disk according to some embodiments of the present disclosure;

FIG. 6B is another schematic diagram illustrating an exemplary water-blocking disk according to some embodiments of the present disclosure;

FIG. 7A is a schematic diagram illustrating an exemplary enclosure according to some embodiments of the present disclosure;

FIG. 7B is a schematic diagram illustrating an exemplary position-limiting member according to some embodiments of the present disclosure;

FIG. 7C is a schematic diagram illustrating an exemplary enclosure and an exemplary envelope according to some embodiments of the present disclosure;

FIG. 7D is another schematic diagram illustrating an exemplary position-limiting member according to some embodiments of the present disclosure;

FIG. 8 is a schematic diagram illustrating an exemplary enclosure, an exemplary envelope, and an exemplary water-blocking disk according to some embodiments of the present disclosure;

FIG. 9A is a schematic diagram illustrating a stirring result using a stirring device before improvement according to some embodiments of the present disclosure; and

FIG. 9B is a schematic diagram illustrating a stirring result using a stirring device after improvement according to some embodiments of the present disclosure.

Accompanying markings include: a bioreactor 100; a tank 101; a reactant 102; a top portion 103 of the tank; a rotating shaft 201; a stirring assembly 202; a magnetic member 203; a connecting member 204; a fixing member 301; a bearing assembly 302; a radial bearing 401; an axial bearing 402; an envelope 501, a vacuuming and liquid-collecting member 502, a separating plate 503; a water-blocking disk 601; an upper surface 601a of the water-blocking disk; a convex loop 602, an annular convex structure 603, and a zigzag channel 604; an enclosure 701; a lower surface 701a of the enclosure, a height h of the enclosure 701, a gap d1 between the enclosure 701 and the envelope 501, a distance d2 between the connecting member 204 and a lower surface of the top portion 103 of the tank, a distance d3 between the envelope 501 and the lower surface of the top portion 103 of the tank, a distance j between the upper surface 601a of the water-blocking disk and the lower surface of the top portion 103 of the tank, a position-limiting member 702, and a locking rib 703.

### DETAILED DESCRIPTION

In order to more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for a person of ordinary skill in the art to apply the present disclosure to other similar scenarios in accordance with these drawings without creative labor. Unless obviously obtained from the context or the context illustrates otherwise, the same numeral in the drawings refers to the same structure or operation.

It should be understood that the terms “system”, “device”, “unit,” and/or “module” as used herein is a method for distinguishing between different components, elements, parts, sections, or assemblies at different levels. However, the words may be replaced by other expressions if other words accomplish the same purpose.

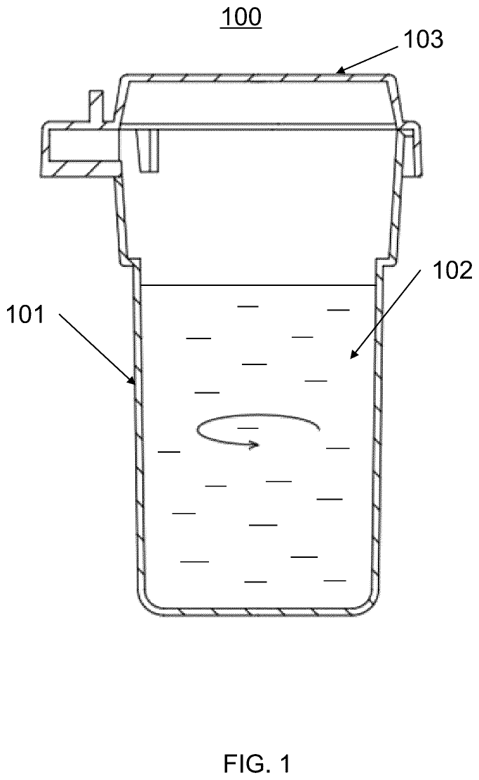

FIG. 1 is a schematic diagram illustrating an exemplary bioreactor according to some embodiments of the present disclosure. As shown in FIG. 1, the bioreactor 100 may include a tank 101 configured for accommodating a reactant 102, and the tank 101 may include a top portion 103 of the tank.

The bioreactor 100 may be used for cell proliferation, monitoring, and maintaining cell physiological status.

The tank 101 refers to a container for accommodating the reactant 102. The reactant 102 may include a mixture of cells to be proliferated as well as a liquid such as a nutrient solution required for cell growth. The composition regarding the reactant is merely exemplary and does not constitute a limitation of the embodiments.

Mixing and stirring of the reactant may be carried out in the bioreactor 100, which allows gases, liquids, and/or even suspended particles in the reactant to be mixed well. A stirring device (not shown in FIG. 1) may be provided for stirring.

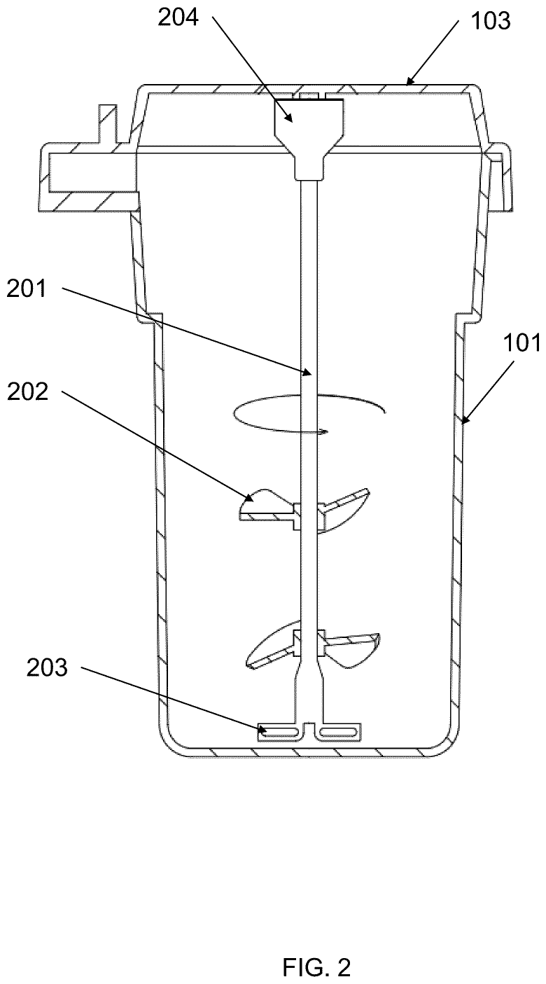

FIG. 2 is a schematic diagram illustrating an exemplary stirring device according to some embodiments of the present disclosure. Some of the following embodiments may be understood with reference to FIG. 2, but the accompanying drawings are only illustrative of some of these embodiments and do not constitute a limitation of the embodiments.

In some embodiments, as shown in FIG. 2, the stirring device may include a rotating shaft 201, a stirring assembly 202, a magnetic member 203, and a connecting member 204. The bottom portion of the rotating shaft 201 may be connected to the magnetic member 203, and the magnetic member 203 may be configured to drive the rotating shaft 201 to rotate without contact. In some embodiments, the rotating shaft 201 may not contact with the bottom portion of the tank 101. The rotating shaft 201 may be connected to the top portion of the tank 101 through the connecting member 204. The magnetic member 203 may be driven by a drive device (not shown in FIG. 2) outside the tank 101, so as to drive the rotating shaft 201 to rotate. The drive device may not contact with the magnetic member 203.

The rotating shaft 201 refers to a shaft for rotation. The stirring assembly 202 (e.g., a paddle or other means) may be connected with the rotating shaft 201 to perform a stirring function. The bottom portion of the rotating shaft 201 refers to a side of the rotating shaft 201 close to the bottom portion of the tank 101. The bottom portion of the tank 101 refers to a side of the tank 101 to which the connecting member 204 is not connected.

In some embodiments, the stirring assembly 202 may be disposed on the rotating shaft 201. When the rotating shaft 201 rotates, the stirring assembly 202 may be driven by the rotating shaft 201 to rotate, which allows gases, liquids, and/or suspended particles in a solution to be mixed evenly.

In some embodiments, the bottom portion of the rotating shaft 201 may be connected with the magnetic member 203. The rotating shaft 201 may not contact with the bottom portion of the tank 101, and the magnetic member 203 may not contact with the bottom portion of the tank 101. For example, the rotating shaft 201 may be suspended from the top portion 103 of the tank in a contact-free manner. Through the contactless design of the rotating shaft 201 and the magnetic member 203 with the bottom portion of the tank 101, it is possible to avoid the occurrence of wear and tear between the tank 101 and at least one of the rotating shaft 201 or the magnetic member 203 which leads to impurities, thereby avoiding the impurities from contaminating the reactant 102 in the tank 101.

The magnetic member 203 refers to an element with magnetic properties. In some embodiments, the material of the magnetic member 203 may include a permanent magnetic material, an electromagnetic material, or the like. In some embodiments, the magnetic member 203 may be fixedly attached to the bottom portion of the rotating shaft 201, e.g., integrally molded, etc. In some embodiments, the magnetic member 203 may be detachably connected with the rotating shaft 201 in a removable connection manner, e.g., a threaded connection manner, a screw connection manner, etc.

In some embodiments, the magnetic member 203 may be driven by a drive device outside of the tank with no contact. In some embodiments, the magnetic member 203 may be driven by the drive device to rotate, and the rotation of the magnetic member 203 may drive the rotating shaft 201 to rotate.

The drive device refers to a device for generating a magnetic field. The drive device may include a drive assembly and a magnet assembly. The drive assembly may be connected to the magnet assembly to drive the magnet assembly to rotate around the axis direction of the rotating shaft 201. The magnet assembly of the drive device may drive the magnetic member 203 connected to the bottom portion of the rotating shaft 201 to rotate by a magnetic force, which in turn causes the magnetic member 203 to drive the rotating shaft 201 to rotate around the axis of the rotating shaft 201. The drive assembly may include a motor or other devices.

In some embodiments, the drive device may not be a part of the bioreactor described in the present disclosure. The descriptions of the drive device are only for illustrations and do not constitute a limitation of the embodiments.

The connecting member 204 refers to a device that provides a connection between the rotating shaft 201 and the tank 101. In some embodiments, the connecting member 204 may be used to fix the rotating shaft 201 and provide a freedom degree of rotation for the rotating shaft 201, thereby enabling the rotating shaft 201 to rotate within the tank 101. In some embodiments, the connecting member 204 may be disposed on the lower surface of the top portion 103 of the tank, and the rotating shaft 201 may be connected to the top portion 103 of the tank via the connecting member 204. The lower surface of the top portion 103 of the tank refers to a surface facing the inside of the tank 101 among two side surfaces of the top portion 103 of the tank.

In some embodiments, the top portion 103 of the tank and the rotating shaft 201 may be detachably connected through the connecting member 204, e.g., a snap connection, a threaded connection, or the like.

In some embodiments, the connecting member 204 may include a first position-limiting member (not shown in FIG. 2) disposed at the top portion 103 of the tank and a second position-limiting member (not shown in FIG. 2) disposed at the top portion of the rotating shaft 201. The first position-limiting member may be connected with the second position-limiting member.

In some embodiments, the first position-limiting member may be a positioning convex or a positioning groove, and the second position-limiting member may be a positioning groove or a positioning convex. The first position-limiting member and the second position-limiting member are removably connected. The first position-limiting member should be matched with the second position-limiting member. For example, when the first position-limiting member is a convex, the second position-limiting member is a groove, and the convex may be movably snapped into the groove to make the top portion 103 of the tank fixed relative to the rotating shaft 201. Relative rotation between the top portion 103 of the tank and the rotating shaft 201 may be achieved by forming a friction pair (e.g., a sliding friction pair or otherwise) between the top portion 103 of the tank and the rotating shaft 201.

In some embodiments, the first position-limiting member and the second position-limiting member may be any other structural form capable of matching with each other to realize a snap-fit connection.

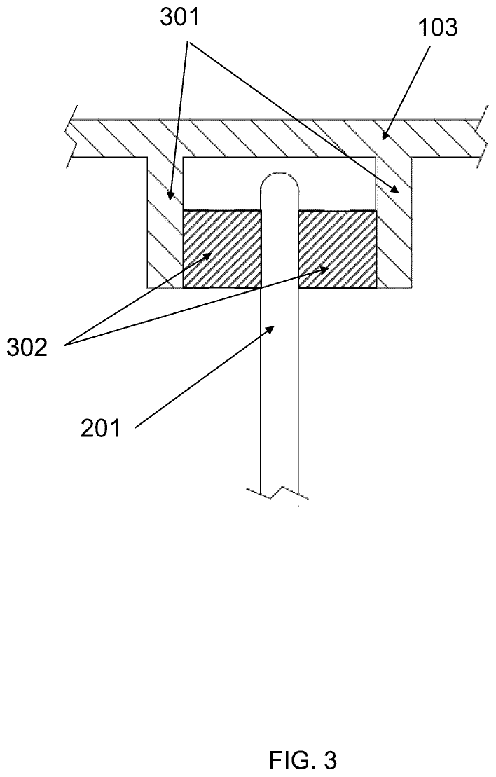

In some embodiments, the connecting member 204 may include a bearing assembly, and the rotating shaft 201 may be connected with the top portion 103 of the tank via the bearing assembly. More descriptions of the bearing assembly may be found in FIG. 3 and the descriptions thereof.

In some embodiments, the connecting member 204 may also include other structural forms. For example, the connecting member 204 may be a rotational structure such as a ball, a roller, a needle, or the like, or a combination thereof. As a further example, the top portion 103 of the tank may be provided with a positioning groove, the connecting member 204 may be a ball structure, and the rotating shaft 201 may be fixed in the positioning groove through the ball structure and connected with the top portion 103 of the tank.

Due to the non-contact setup of the rotating shaft and the bottom portion of the tank, wear and tear of the structure due to the existence of the connecting structure between the rotating shaft and the bottom portion of the tank may be reduced or avoided, and the generation of impurities contaminating liquid caused by the wear and tear of the structure may be further avoided, henceforth reducing the risk of the liquid being contaminated. At the same time, the structure of the bioreactor may be simplified and the cost of the production and use of the bioreactor, which a rotational speed and may be effectively increased and the service life of the stirring device may be prolonged.

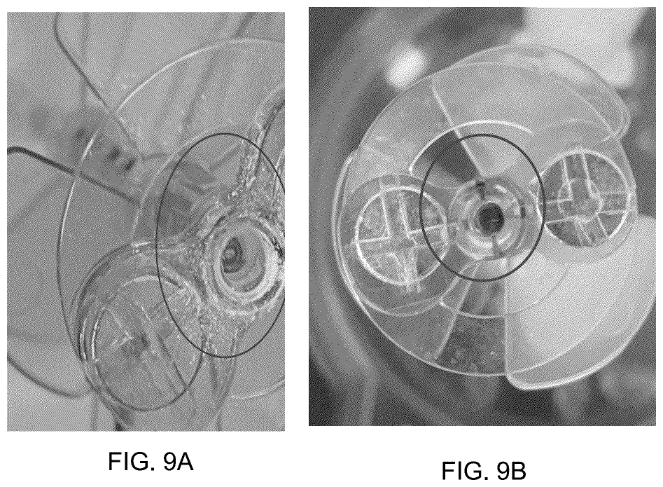

FIG. 9A is a schematic diagram illustrating a stirring result using a stirring device before improvement. The stirring device before improvement includes a rotating shaft fixed to a top portion of a tank by a fixed sleeve, a groove disposed at a bottom portion of the rotating shaft, a column disposed at a bottom portion of the tank. And the rotating shaft and the bottom portion of the tank are connected by the meshing of the groove and the column. A large friction may be generated at a place where the fixed sleeve is in contact with the rotating shaft and at a place where the bottom portion of the rotating shaft is in contact with the bottom portion of the tank during stirring. As a result, the stirring device before improvement produces a large number of powdery particles under rotational friction. As shown in FIG. 9A, a large number of powdery particles was generated at the place where the fixed sleeve is in contact with the rotating shaft and at the place where the bottom portion of the rotating shaft is in contact with the bottom portion of the tank during stirring.

FIG. 9B is a schematic diagram illustrating a stirring result using a stirring device after improvement according to some embodiments of the present disclosure. As shown in FIG. 9B, the bottom portion of a tank no longer produces powdery particles. The stirring device as described in the present disclosure a rotating shaft suspended and fixed at a top portion of the tank through a connecting member, and when stirring, wear and tear may be avoided due to the existence of a connection between the rotating shaft and the bottom portion of the tank, thereby reducing or eliminating a friction generated at the bottom portion of the rotating shaft.

FIG. 3 is a schematic diagram illustrating an exemplary connecting member according to some embodiments of the present disclosure. Some of the following embodiments may be understood with reference to FIG. 3, but the accompanying drawings are only a schematic illustration of some of these embodiments, and do not constitute a limitation of the embodiments.

As shown in FIG. 3, the connecting member 204 may include the bearing assembly 302, and the bearing assembly 302 may be connected to the tank 101 (not shown in FIG. 3) through the fixing member 301. The fixing member 301 may be an integral part of the tank 101, the fixing member 301 may be disposed on the lower surface of the top portion 103 of the tank, and the fixing member 301 may be not in contact with the rotating shaft 201.

The fixing member 301 may be a member for fixing the connecting member 204.

In some embodiments, the fixing member 301 may be a tubular structure and the bottom portion of the fixing member 301 may be connected to a stationary portion (e.g., a bearing outer ring) of the bearing assembly 302, a rotating portion (e.g., a bearing inner ring) of the bearing assembly 302 may be connected to the rotating shaft 201, the top end of the rotating shaft 201 may pass through a center hole of the bearing assembly 302 and extend into the interior of the fixing member 301, the top end of the rotating shaft 201 may be not in contact with the top portion 103 of the tank, and there may be a gap between the peripheral side of the rotating shaft 201 and the bearing assembly 302 and the fixing member 301. The top portion of the rotating shaft 201 refers to a side of the rotating shaft 201 close to the top portion of the tank 101.

In some embodiments, the fixing member 301 may be in other structural forms. For example, the fixing member 301 may be a columnar structure that is connected to the bearing assembly 302. At this point, the top portion of the rotating shaft 201 may extend into the center hole of the bearing assembly 302 but is not in contact with the fixing member 301. The fixing member 301 may be a columnar structure with a projection at the bottom portion and the projection may extend into the center hole of the bearing assembly 302 while the rotating shaft 201 may not extend into the center hole of the bearing assembly 302, and the top portion of the rotating shaft 201 may not in contact with the fixing member 301. The fixing member 301 may be any other structure that is connected to a stationary portion of the bearing assembly and is not in contact with the rotating shaft.

In some embodiments, the fixing member 301 may provide an upward support force for the rotating shaft 201 via the bearing assembly 302. For example, the fixing member 301 may be connected with the stationary portion of the bearing assembly 302, and the rotating portion of the bearing assembly 302 may be connected to the rotating shaft. The gravity force of the rotating shaft 201 may be transferred through the bearing assembly 302 to the fixing member 301, and the fixing member 301 may provide the upward support force for the rotating shaft 201.

The bearing assembly 302 may include a bearing that provides a connection between the rotating shaft and the tank. The bearing assembly 302 may be connected with the fixing member 301 by welding, bolting, or the like.

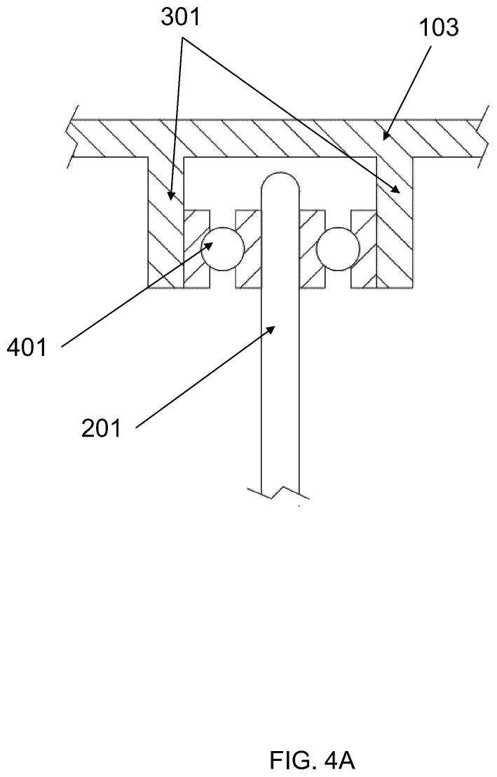

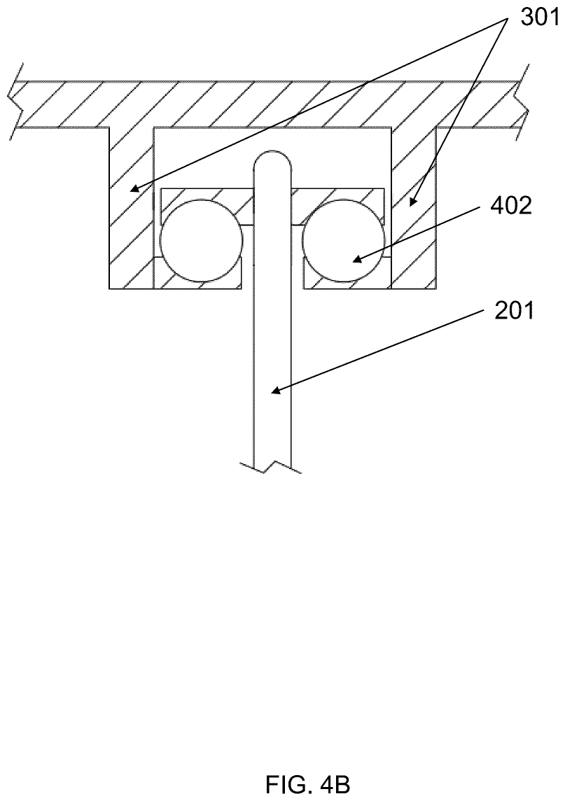

In some embodiments, the bearing assembly 302 may include a radial bearing. FIG. 4A is a schematic diagram illustrating an exemplary connecting member including a bearing assembly that includes a radial bearing according to some embodiments of the present disclosure. Some of the following embodiments may be understood with reference to FIG. 4A. However, the accompanying drawings are only schematic illustrations of some of these embodiments and do not constitute a limitation of the embodiments.

In some embodiments, as shown in FIG. 4A, the fixing member 301 may be connected with the outer ring of the radial bearing 401 and the outer ring of the radial bearing 401 may be a stationary portion of the radial bearing 401, and the rotating shaft 201 may be connected with the inner ring of the radial bearing 401 and the inner ring of the radial bearing 401 may be a rotating portion of the radial bearing 401.

In some embodiments, the fixing member 301 may be connected with the inner ring of the radial bearing 401 and the inner ring of the radial bearing 401 may be a stationary portion; and the rotating shaft 201 may be connected to the outer ring of the radial bearing 401 and the outer ring of the radial bearing 401 may be a rotating portion.

In some embodiments, the bearing assembly 302 may include an articular bulb bearing. The fixing member may be connected with the inner spherical surface of the articular bulb bearing, and the rotating shaft may be connected with the outer spherical surface of the articular bulb bearing.

In some embodiments, the bearing assembly 302 may include an axial bearing, and the axial bearing may be able to withstand an axial load applied by the rotating shaft 201. FIG. 4B is a schematic diagram illustrating an exemplary connecting member including a bearing assembly that includes an axial bearing according to some embodiments of the present disclosure. Some of the following embodiments may be understood with reference to FIG. 4B. However, the accompanying drawings are only schematic illustrations of some of the embodiments therein, and do not constitute a limitation of the embodiments.

In some embodiments, as shown in FIG. 4B, the axial bearing 402 may include an upper ring and a lower ring, and the upper ring may be a rotating portion and the lower ring may be a stationary portion of the axial bearing 402. Inner diameters of the upper ring and lower ring are different, and the inner diameter of the upper ring is smaller than the inner diameter of the lower ring. The fixing member 301 may be connected with the lower ring of the axial bearing 402, and the rotating shaft 201 may pass through the center hole of the axial bearing 402 and may be connected with the upper ring of the axial bearing 402. The upper ring of the axial bearing 402 refers to a ring body on one side close to the top portion 103 of the tank.

In some embodiments, the bearing assembly 302 may include a roller bearing. The roller bearing may withstand both axial and radial loads provided by a centrifugal force. For example, the roller bearing may withstand an axial force applied by the rotating shaft 201.

In some embodiments, the bearing assembly may be in any other feasible form and is not limited herein.

The rotating shaft may be hung on the fixing member through the bearing assembly, so that a part or all of the weight of the rotating shaft is supported by the fixing member and the rotating shaft, which is capable of reducing or eliminating a friction generated at the bottom portion of the rotating shaft, henceforth effectively improving a stirring speed. At the same time, such a connection manner can reduce particles generated by a friction when the rotating shaft rotates, and reduce the contamination of the particles to other parts inside the tank. The top portion of the rotating shaft is restrained by the bearing assembly, which can reduce the degree of shaking when the rotating shaft rotates.

In some embodiments, the fixing member 301 may be connected with the bearing assembly 302 via a telescoping member (not shown in FIG. 3). The telescoping member may be in a variety of forms, e.g., a spring, etc. In some embodiments, the telescoping state of the telescoping member may be changed in response to a magnetic force. For example, under a zero magnetic force, the telescoping member may be in a stretched state under the gravity of the rotating shaft 201, the stirring assembly 202, the magnetic member 203, or the like, or a combination thereof, and the telescoping member may have a shortest telescoping length. Under the magnetic force, the telescoping member may be stretched further under the gravity of the rotating shaft 201, the stirring assembly 202, the magnetic member 203, or the like, as hole as the magnetic force. As the magnitude of the magnetic force is changed, the telescoping length of the telescoping member may be synchronized to be changed, which in turn changes the telescoping state of the telescoping member.

By adjusting the telescoping state of the telescoping member, a distance between the fixing member 301 and the bearing assembly 302 may be adjusted, and thus heights of the rotating shaft 201, the stirring assembly 202, and the magnetic member 203 may be adjusted, so as to adjust a stirring height.

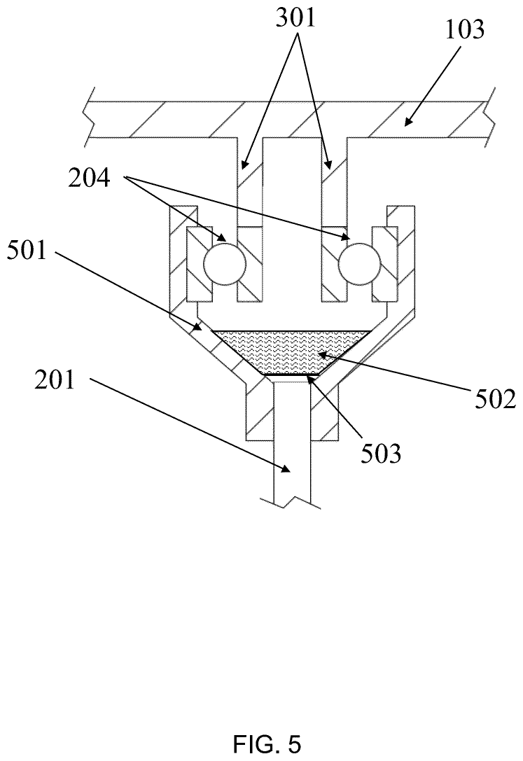

FIG. 5 is a schematic diagram illustrating an exemplary envelope according to some embodiments of the present disclosure. Some of the following embodiments may be understood with reference to FIG. 5, but the accompanying drawings are illustrative of only some of these embodiments and do not constitute a limitation of the embodiments.

In some embodiments, as shown in FIG. 5, the stirring device may further include an envelope 501. The envelope 501 may be disposed around the connecting member 204. The lower end of the envelope 501 may be fixedly connected with the rotating shaft 201 with no gap, and the upper end of the envelope 501 may be connected with the connecting member 204.

The envelope 501 refers to a kit that seals the connecting member 204. The envelope 501 may include a variety of structural forms. For example, the envelope 501 may include a cylinder surrounding the periphery of the connecting member 204, and a cone disposed below and connected with the cylinder. The cone may have a conical apex underneath and being fixedly connected with the rotating shaft 201 with no gap. As another example, the envelope 501 may be a sphere with an opening at the top that encases the connecting member 204 and the rotating shaft 201 within the envelope 501. The bottom portion of the spherical shape may be connected with the rotating shaft 201 with no gap. In some embodiments, the shape of the envelope 501 may have other variations.

In some embodiments, the envelope 501 may be a funnel-shaped structure, and the connecting member 204 may be disposed within an opening at the top portion of the envelope 501. The connecting member 204 may be fixedly connected with an inner side of an upper end of the envelope 501, the rotating shaft 201 may be inserted through a through-hole at the lower end of the envelope 501, and the outer peripheral side of the rotating shaft 201 may be connected with the through-hole at the lower end of the envelope 501 with no gap. The upper end of the envelope 501 refers to a side close to the top portion 103 of the tank. The lower end of the envelope 501 refers to a side away from the top portion 103 of the tank.

In some embodiments, the upper end of the envelope 501 may be disposed around the connecting member 204, and the connecting member 204 may be disposed within a space surrounded by the envelope 501. The upper end of the envelope 501 may be connected with the connecting member 204. In some embodiments, the lower end of the envelope 501 may be disposed around the rotating shaft 201, and the lower end of the envelope 501 may be connected with the rotating shaft 201 with no gap. Manners of a connection between the upper end of the envelope 501 and the connecting member 204 and/or between the lower end of the envelope 501 and the rotating shaft 201 may include but are not limited to, snap-fitting, welding, bolting, or the like. The connection of the lower end of the envelope 501 and the connecting member 204 allows the envelope 501 to drive at least a portion of the connecting member 204 to rotate. In some embodiments, the lower end of the envelope 501 may be connected with the rotating shaft 201 with no gap, and impurities such as particles generated by a friction of the connecting member 204 may fall within the envelope 501.

In some embodiments, when the rotating shaft 201 is inserted through the through-hole at the lower end of the envelope 501, the top portion of the rotating shaft 201 may be aligned with the through-hole at the lower end of the envelope 501. In some embodiments, when the rotating shaft 201 is inserted through the through-hole at the lower end of the envelope 501, the top portion of the rotating shaft 201 may extend into the interior of the envelope 501 but not extend to the connecting member 204. In some embodiments, when the rotating shaft 201 is inserted through the through-hole at the lower end of the envelope 501, the top portion of the rotating shaft 201 may extend to a center hole of the connecting member 204.

In some embodiments, the rotating shaft 201 and the lower end of the envelope 501 may be integrally molded such that the rotating shaft 201 may be fixedly connected to the through-hole at the lower end of the envelope 501 with no gap. In some embodiments, the rotating shaft 201 and the lower end of the envelope 501 may be fixedly connected such that the rotating shaft 201 may be fixedly connected to the through-hole at the lower end of the envelope 501 with no gap.

In some embodiments, the distance between an upper surface of the envelope 501 and a lower surface of the top portion 103 of the tank may range from 0.5-1.5 mm. In some embodiments, the distance between the upper surface of the envelope 501 and the lower surface of the top portion 103 of the tank may be 0.5 mm. In some embodiments, the distance between the upper surface of the envelope 501 and the lower surface of the top portion 103 of the tank may be less than a distance threshold. The upper surface of the envelope 501 refers to a surface of the envelope 501 close to the top portion 103 of the tank. The distance threshold may be a system default value, an empirical value, a human pre-set value, etc., or any combination thereof, and may be set according to actual needs, which is not limited herein.

In some embodiments, the connecting member 204 may include the bearing assembly 302, and the envelope 501 may be disposed to wrap around the bearing assembly 302. More descriptions of the bearing assembly can be found in FIG. 3 and description thereof.

In some embodiments, there may be a spacing between the envelope 501 and the fixing member 301. For example, the spacing may exist between an inner sidewall of the upper end of the envelope 501 and an outer sidewall of the fixing member 301. In some embodiments, the length of the spacing may be in a range of 2 to 4 mm. In some embodiments, the length of the spacing may be 3 mm.

In some embodiments, when the bearing assembly 302 includes the radial bearing, the rotating shaft 201 may be connected with an outer ring of the radial bearing, and an inner ring of the radial bearing may be connected with the fixing member 301. At this point, the inner sidewall of the envelope 501 may be connected with the outer ring of the radial bearing and wrap around the radial bearing. The envelope 501 may be driven to rotate by the rotating shaft 201, the envelope 501 may in turn drive the outer ring of the radial bearing to rotate, and the inner ring of the radial bearing may be fixed to the fixing member 301. At this time, the torque of the rotating shaft 201 may be transmitted to the outer ring of the radial bearing.

In some embodiments, when the bearing assembly 302 includes a radial bearing, the rotating shaft 201 may be connected with the inner ring of the radial bearing, and the outer ring of the radial bearing may be connected with the fixing member 301. At this time, an upper portion of the envelope 501 may be disposed around the radial bearing but is not connected to the radial bearing, and the torque of the rotating shaft 201 may be transmitted directly to the inner ring of the radial bearing.

In some embodiments, the interior of the envelope 501 may include an unoccupied space. In some embodiments, the unoccupied space may be enclosed by the envelope 501 and the connecting member 204 and is unoccupied by the rotating shaft 201, which may be used to accommodate dropped particles, splashed liquids, or the like.

The envelope 501 may collect particles, splashing liquids, etc., generated by a rotating friction to reduce the contamination of the particles and the liquids to the rest inside the tank.

In some embodiments, as shown in FIG. 5, the vacuuming and liquid-collecting member 502 is disposed inside the envelope 501, and the vacuuming and liquid-collecting member 502 is connected with a bottom inside the envelope 501.

The vacuuming and liquid-collecting member 502 may be a member for adsorption and collection of impurities. The vacuuming and liquid-collecting member 502 may be in various forms. For example, the vacuuming and liquid-collecting member 502 may include a cotton, a sponge, etc.

In some embodiments, at least one opening may be disposed in the vacuuming and liquid-collecting member 502 to accommodate dropped particles. In some embodiments, a plurality of openings provided in the vacuuming and liquid-collecting member 502 may be in a form of vertical holes, round holes, or other forms.

In some embodiments, there may be a spacing between the vacuuming and liquid-collecting member 502 and the connecting member 204 to avoid interference of the vacuuming and liquid-collecting member 502 to the connecting member 204.

In some embodiments, a separating plate 503 may be provided at a bottom portion inside the envelope 501, the separating plate 503 may be configured to separate the vacuuming and liquid-collecting member 502 from the top portion of the rotating shaft 201. In some embodiments, the vacuuming and liquid-collecting member 502 may be disposed above the separating plate 503, and the top portion of the rotating shaft 201 may be located below the separating plate 503.

In some embodiments, a spacing may exist between the separating plate 503 and the top portion of the rotating shaft 201 to avoid the vacuuming and liquid-collecting member 502 from interfering with the rotation of the rotating shaft 201. In some embodiments, when the separating plate 503 is disposed at the bottom portion inside the envelope 501, the top portion of the rotating shaft 201 may be aligned with the through-hole at the lower end of the envelope 501, or the top portion of the rotating shaft 201 may extend into the interior of the envelope 501 but not extend to the connecting member 204. At this time, there may be a spacing between the top portion of the rotating shaft 201 and the separating plate 503.

By setting the vacuuming and liquid-collecting member 502 inside the envelope 501, particles and condensation water generated by the stirring device during rotation, oil leaking from the bearing assembly, etc., can be absorbed, and such impurities can be prevented from falling into the bottom portion of the tank to contaminate materials. At the same time, it can effectively prevent liquids and particulate impurities in the envelope from being thrown out of the envelope by a centrifugal action when the stirring device rotates (or in extreme cases, only the liquids are thrown out of the envelope and the particulate impurities are adsorbed by the vacuuming and liquid-collecting member), thus increasing the service life of the stirring device. By setting holes in the vacuuming and liquid-collecting member 502, particulate impurities can be effectively avoided from being deposited solely on a surface of the vacuuming and liquid-collecting member 502, and the service life of the vacuuming and liquid-collecting member 502 can be increased.

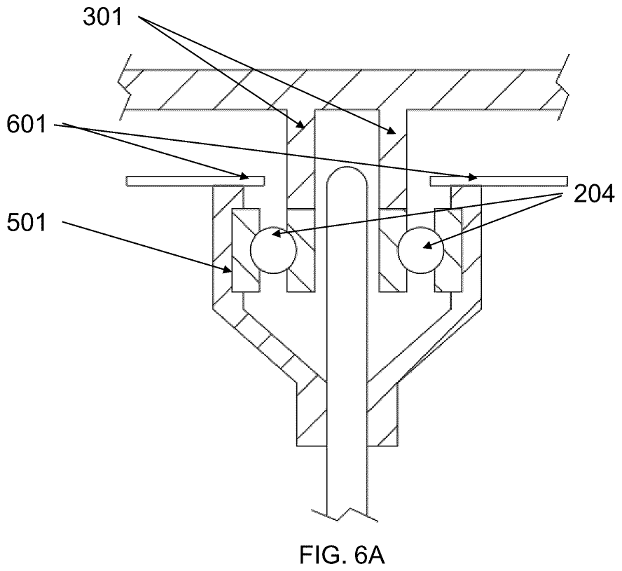

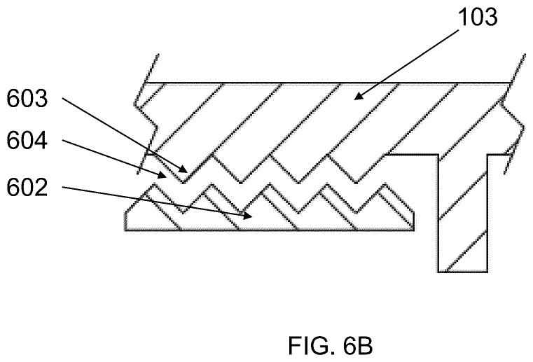

FIG. 6A is a schematic diagram illustrating an exemplary water-blocking disk according to some embodiments of the present disclosure. FIG. 6B is another schematic diagram illustrating an exemplary water-blocking disk according to some embodiments of the present disclosure. Some of the following embodiments may be understood with reference to FIG. 6A and FIG. 6B, but the accompanying drawings are only illustrative of some of these embodiments and do not constitute a limitation of the embodiments.

In some embodiments, the top portion of the envelope 501 may be provided with a water-blocking disk 601. The water-blocking disk 601 may be disposed above the envelope 501, and the bottom portion of the water-blocking disk 601 may be connected with the top portion of the envelope 501.

In some embodiments, the water-blocking disk 601 may be an integral part of the envelope 501, and the water-blocking disk 601 may be integrally molded with the envelope 501.

The water-blocking disk 601 refers to a structure that blocks water from entering the envelope 501. In some embodiments, the water-blocking disk 601 may be disposed around the fixing member 301.

In some embodiments, the water-blocking disk 601 may be a structure having a through-hole in the center, through which the fixing member 301 may extend into the interior of the envelope 501 and be connected with the connecting member 204. In some embodiments, the shape of the through-hole in the center of the water-blocking disk 601 may be similar to a cross-section shape of the fixing member 301. The fixing member 301 may be an integral part of the tank 101, and the fixing member 301 may be disposed on a lower surface of the top portion 103 of the tank. Further description of the fixing member 301 can be found in FIG. 3 and the descriptions thereof.

The water-blocking disk 601 may include a variety of structural forms. For example, the water-blocking disk 601 may be a disk-like structure with a through-hole in the center. The water-blocking disk 601 and the envelope 501 may together enclose a space enclosing the connecting member 204. For example, the water-blocking disk 601 may be a planar disk and have a through-hole in the center region. As another example, the water-blocking disk 601 may be a conical-like structure. The center region of the water-blocking disk 601 may be higher than the rim region of the water-blocking disk 601. The water-blocking disk 601 and the envelope 501 may enclose a space with a conical top a through-hole may be provided in the center region of the water-blocking disk 601. In some embodiments, the water-blocking disk 601 may also be other structures.

The water-blocking disk may be located above the connecting member and the envelope. The bottom portion of the water-blocking disk may be connected to the top portion of the envelope, enclosing a space for accommodating particles generated by a friction.

In some embodiments, the rim region of the water-blocking disk 601 may extend out of the top portion of the envelope 501 to form a cornice-like structure, which is capable of preventing liquids outside of the envelope 501 from splashing during a stirring process and entering the interior of the envelope 501.

In some embodiments, there may be no holes on a disk surface of the water-blocking disk 601. In some embodiments, the water-blocking disk 601 may be inclinedly disposed above the top portion of the envelope 501. In some embodiments, the center region of the water-blocking disk 601 may be higher than the rim region of the water-blocking disk 601, i.e., a side of the water-blocking disk 601 that is proximate to the fixing member 301 or the rotating shaft 201 is higher than the rim region of the water-blocking disk 601. The center region of the water-blocking disk is higher than the rim region of the water-blocking disk, that is, a distance between the center region of the water-blocking disk and the lower surface of the top portion of the tank is smaller than a distance between the rim region of the water-blocking disk and the lower surface of the top portion of the tank. In some embodiments, a disk surface of the water-blocking disk 601 may be provided with a concentric circular groove, a surface of the groove being lower than two rim regions of the water-blocking disk.

By setting the water-blocking disk at an incline, reactants splashing onto the water-blocking disk can be prevented from falling into the connecting member and be allowed to fall back into the tank.

In some embodiments, the lower surface of the top portion 103 of the tank may include one or more annular convex structures, the one or more annular convex structures may have different diameters and be co-circular. The upper surface of the water-blocking disk 601 may include a convex loop, the convex loop may include one or more concentric rings of different diameters. The convex loop may be embedded in the one or more annular convex structures.

The upper surface of the water-blocking disk 601 refers to a surface on the water-blocking disk 601 near the top portion 103 of the tank. The convex loop may be a convex structure including a plurality of concentric rings that have different diameters.

In some embodiments, a concentric ring in the p convex loop may be embedded in a gap between two adjacent annular convex structures.

In some embodiments, the convex loop may be in no contact with the one or more annular convex structures along a radial direction of the tank such that a zigzag channel is formed between the convex loop and the one or more annular convex structures.

The zigzag channel may be a gap between the convex loop and the plurality of annular convex structures. In some embodiments, a gap between each two adjacent annular convex structures may be used to embed a concentric ring, and there is a gap between the concentric ring and the annular convex structures along an axial direction of the tank, i.e., no contact between the concentric ring and the annular convex structures. Gaps between a plurality of concentric rings of the convex loop and the plurality of annular convex structures may form the zigzag channel. Because of the zigzag channel, it is difficult for condensate and water vapor to enter the interior of the envelope.

Some of the following embodiments may be understood with reference to FIG. 6B. However, the accompanying drawings are only schematic illustrations of some of these embodiments and do not constitute a limitation of the embodiments.

The upper surface of the water-blocking disk 601 may have the convex loop 602, and the lower surface of the top portion 103 of the tank may include at least one convex structure 603. The convex loop 602 and the at least one convex structure 603 may be concentric and have corresponding shapes. Gaps between the convex loop 602 and the at least one convex structure 603 may form the zigzag channel 604. When the water-blocking disk 601 and the top portion 103 of the tank rotate relative to each other, the convex loop 602 only rotates around its axis (i.e., an axis of the rotating shaft) and does not move along the radial direction or axial direction of the convex loop 602.

By setting up the zigzag channel, the path of moisture can be extended, thus reducing or preventing the moisture in the tank from entering the interior of the envelope.

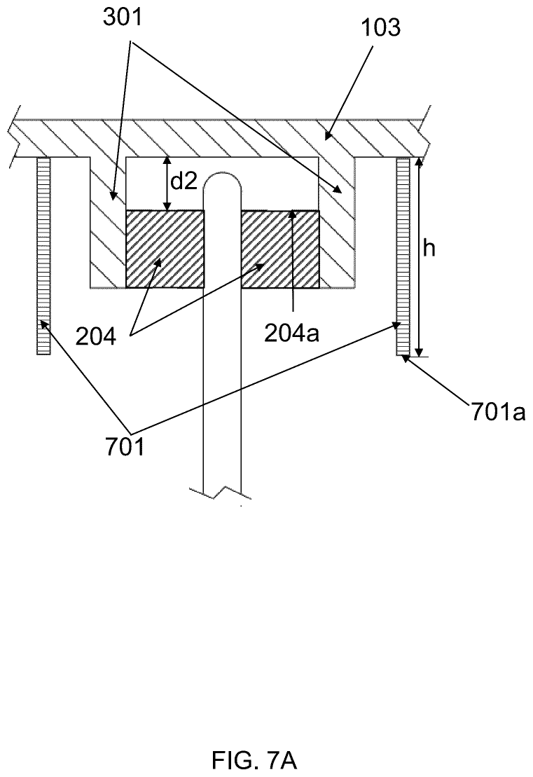

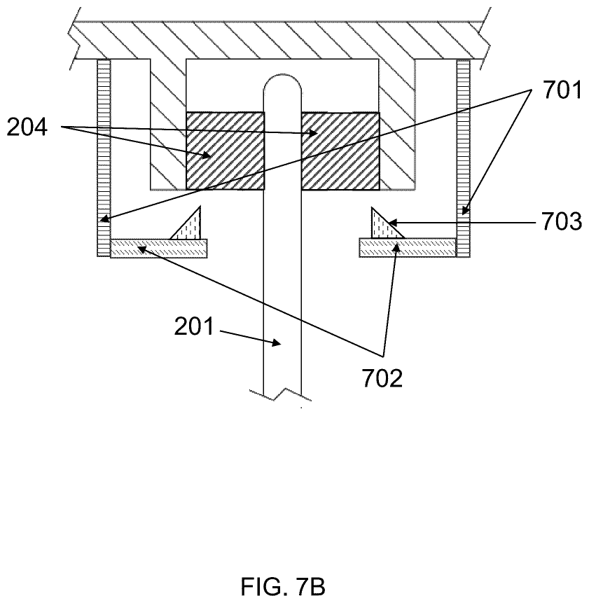

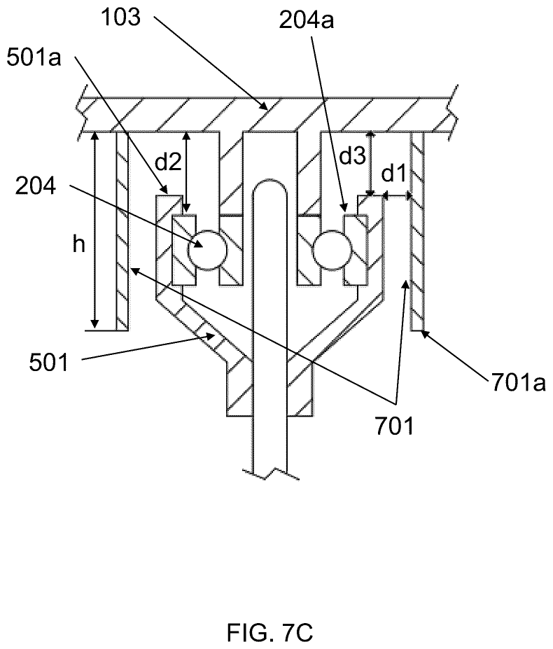

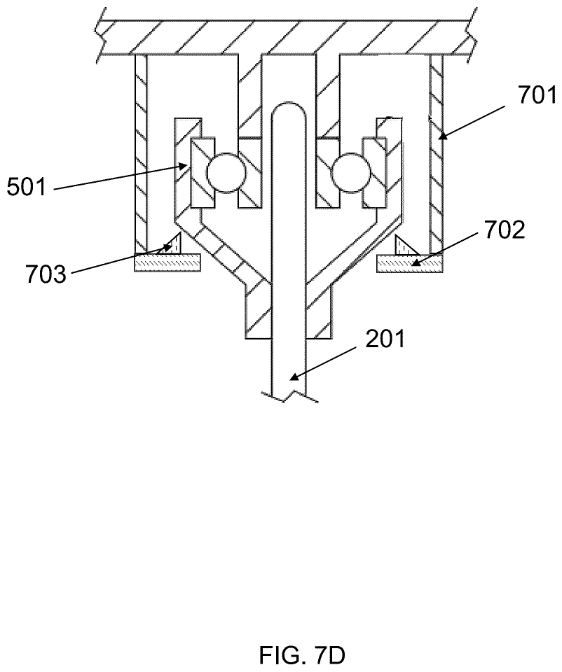

FIG. 7A is a schematic diagram illustrating an exemplary enclosure according to some embodiments of the present disclosure. Some of the following embodiments may be understood with reference to FIG. 7A. However, the accompanying drawings are illustrative of only some of these embodiments and do not constitute a limitation of the embodiments. The lower surface of the enclosure 701 refers to a side surface of the enclosure 701 facing the inside of the tank 101.

In some embodiments, as shown in FIG. 7A, the tank 101 may further include an enclosure 701 disposed below the top portion of the tank. The enclosure 701 may be connected with the lower surface of the top portion 103 of the tank, and the enclosure 701 may be disposed at least partially around the connecting member 204.

The enclosure 701 refers to a protective structure surrounding the outer side of the connecting member 204. The enclosure 701 may be connected with the top portion 103 of the tank.

The enclosure 701 may include a variety of structural forms. For example, the enclosure 701 may include a sleeve structure capable of surrounding the outer side of the connecting member 201. As a further example, the enclosure 701 may be a rotary body-type structure with openings at both ends, e.g., a cylindrical structure, or a circular platform structure. As still a further example, the enclosure 701 may be a prismatic structure with openings at both ends. In some embodiments, the shape of the enclosure 701 may also have various other variations.

The enclosure 701 may be connected with the top portion 103 of the tank by bonding, welding, screwing or threading, etc., or the enclosure 701 may be integrally molded with the top portion 103 of the tank.

The enclosure 701 may be vertically connected with the top portion 103 of the tank or may be set inclinedly relative to the top portion 103 of the tank. In some embodiments, the enclosure 701 may be designed as a cylindrical structure with openings at both ends and connected with the lower surface of the top portion 103 of the tank. In some embodiments, the axial direction of the enclosure 701 may be perpendicular to the lower surface of the top portion 103 of the tank. In some embodiments, the enclosure 701 may be designed as a circular platform structure with openings at both ends and connected with the lower surface of the top portion 103 of the tank inclinedly.

In some embodiments, the height of the enclosure 701 may be greater than a distance between the upper surface of the connecting member 204 and the lower surface of the top portion 103 of the tank. The height of the enclosure 701 refers to a vertical distance between the lower surface of the enclosure 701 and the lower surface of the top portion 103 of the tank. The upper surface of the connecting member 204 refers to a surface of the connecting device 204 close to the top portion 103 of the tank. As shown in FIG. 7A, the lower surface 701a of the enclosure 701 needs to be located below the upper surface 204a of the connecting member, i.e., the height h of the enclosure 701 needs to be greater than a distance d2 between the upper surface 204a of the connecting member and the lower surface of the top portion 103 of the tank.

By setting the enclosure around the connecting member at the top portion of the tank, it can effectively prevent splashing liquids or water vapor from entering the interior of the connecting member when the liquids inside the tank are agitated, avoiding contaminating an interior of a bioreactor.

FIG. 7B is a schematic diagram illustrating an exemplary position-limiting member according to some embodiments of the present disclosure. Some of the following embodiments may be understood with reference to FIG. 7B. However, the accompanying drawings are only illustrative of some of these embodiments and do not constitute a limitation of the embodiments.

In some embodiments, as shown in FIG. 7B, the bottom portion of the enclosure 701 may be provided with a position-limiting member 702. A radial distance between the position-limiting member 702 and the rotating shaft 201 along a radial direction of a tank may be less than a radial width of the connecting member 204 along the radial direction of the tank.

The position-limiting member 702 may be a member for limiting the position of the connecting member 204. In some embodiments, the position-limiting member 702 may be of various structural forms. For example, the position-limiting member 702 may be in a form of a bar, a groove, a plane, or many other forms.

In some embodiments, the position-limiting member 702 may be connected with the enclosure 701 by bonding, welding, screwing, or threading, etc., or the position-limiting member 702 may be integrally molded to the enclosure 701.

By providing the position-limiting member, it is possible to catch the connecting member in case of an accidental fall of a stirring device from the top portion of the tank and limit a falling height of the connecting member, thereby preventing the connecting member from falling into reactants inside the tank and contaminating the reactants. For example, if a centrifugal force is too great when a rotational speed is too large and the centrifugal force causes the stirring device to fall off, or if the connecting member (e.g. a bearing assembly) fails and causes the stirring device to fall off, etc., the stirring device can be jammed to avoid causing contamination.

In some embodiments, as shown in FIG. 7B, the position-limiting member 702 may include one or more locking ribs 703. In some embodiments, the one or more locking ribs 703 may be distributed in an array with a center of a circle of the enclosure 701.

A locking rib 703 is a member used to restrict movement of the stirring device. When the stirring device contacts the position-limiting member in the event of an accidental descent of the stirring device from the top portion of the tank, the stirring device may stop rotating based on a resistance provided by the locking rib 703. The locking rib 703 may include a variety of structural forms, e.g., a triangular rib, a trapezoidal rib, a bladed rib, an edged rib, a semicircular rib, or the like.

In some embodiments, the locking ribs 703 may be disposed in an array in the position-limiting member 702. In some embodiments, a locking rib 703 may be connected with the position-limiting member 702 by bonding, welding, screwing, or threading, or the locking rib 703 may be integrally molded with the position-limiting member 702.

By installing the locking rib on the position-limiting member, it is possible to effectively prevent the stirring device from continuing to rotate after it has been accidentally dislodged from the top portion of the tank, leading to wear and contamination.

FIG. 7C is a schematic diagram illustrating an exemplary enclosure and envelope according to some embodiments of the present disclosure. Some of the following embodiments may be understood with reference to FIG. 7C. However, the accompanying drawings are illustrative of only some of these embodiments and do not constitute a limitation of the embodiments.

In some embodiments, as shown in FIG. 7C, the outside of the connecting member 204 may be surrounded by the envelope 501, and the tank 101 may include the enclosure 701 disposed underneath the top portion of the tank 101. The enclosure 701 may be connected with the lower surface of the top portion 103 of the tank, and at least a portion of the enclosure 701 may be provided around the envelope 501.

In some embodiments, there may be a spacing between the enclosure 701 and the envelope 501 to avoid the enclosure 701 interfering with the rotation of the envelope 501. For example, there is the gap d1 between the enclosure 701 and the envelope 501.

In some embodiments, as shown in FIG. 7C, the height of the enclosure 701 may be greater than the distance between the upper surface of the envelope 501 and the lower surface of the top portion 103 of the tank. That is, the lower surface 701a of the enclosure needs to be located below the upper surface 501a of the envelope. That is, the height h of the enclosure 701 needs to be greater than the distance d3 between the upper surface 501a of the envelope and the lower surface of the top portion 103 of the tank.

By setting the enclosure around the envelope at the top portion of the tank, it can effectively prevent splashing liquids or vapor from entering into the interior of the envelope or the connecting member inside the envelope when the liquids inside the tank are stirred, so as to avoid contaminating the interior of a bioreactor.

FIG. 7D is another exemplary schematic diagram illustrating an exemplary position-limiting member according to some embodiments of the present disclosure. Some of the following embodiments may be understood with reference to FIG. 7D. However, the accompanying drawings are only schematic illustrations of some of these embodiments, and do not constitute a limitation of the embodiments.

In some embodiments, as shown in FIG. 7D, when the outside of connecting member 204 is surrounded by the envelope 501, the enclosure 701 may be provided with the position-limiting member 702 on the bottom portion of the enclosure 701, and a radial distance between the position-limiting member 702 and the rotating shaft 201 along a radial direction of the tank may be less than a radial width of the connecting member 204 along the radial direction of the tank. In some embodiments, the position-limiting member 702 may include the locking ribs 703 distributed in an array with the center of the circle of the enclosure 701, as shown in FIG. 7D. For more description of the position-limiting member 702 and the locking rib 703, refer to FIG. 7B and related descriptions thereof.

By providing the position-limiting member, it is possible to prevent the connecting member from falling into reactants inside the tank and contaminating the reactants by catching the envelope in the event of an accidental fall of a stirring device from the top portion of the tank and thereby limiting a falling height of the connecting member.

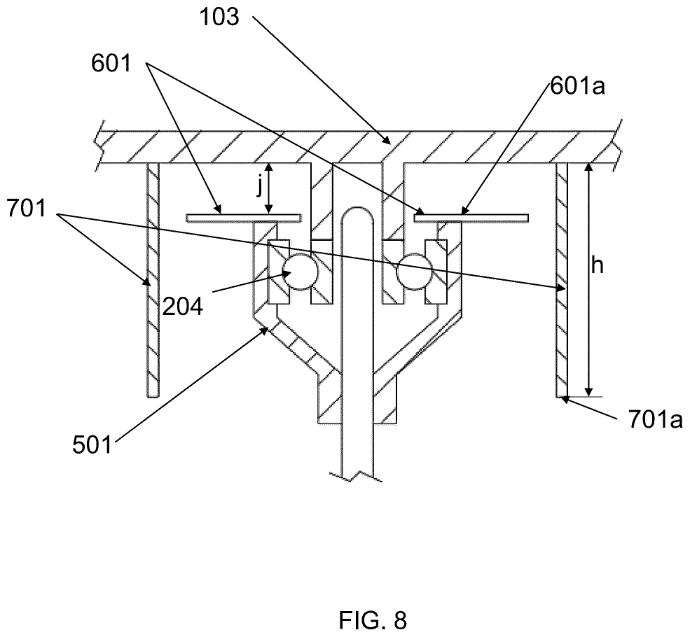

FIG. 8 is a schematic diagram illustrating an exemplary enclosure, an exemplary envelope, and an exemplary water-blocking disk according to some embodiments of the present disclosure. Some of the following embodiments may be understood with reference to FIG. 8, but the accompanying drawings are illustrative of only some of these embodiments and do not constitute a limitation of the embodiments.

In some embodiments, as shown in FIG. 8, the outside of the connecting member 204 may be surrounded by the envelope 501, the envelope 501 is provided with the water-blocking disk 601 on a top, and the enclosure 701 may be provided around the outer side of the envelope 501 and the water-blocking disk 601. The enclosure 701 may be connected with the top portion 103 of the tank. In some embodiments, there may be a certain spacing between each two of the enclosure 701, the envelope 501, and the water-blocking disk 601 to avoid the enclosure interfering with the rotation of the envelope 501 and the water-blocking disk 601.

In some embodiments, the lower surface 701a of the enclosure needs to be located at least below the upper surface 601a of the water-blocking disk, i.e., the height h of the enclosure 701 needs to be greater than the distance j between the upper surface 601a of the water-blocking disk and the lower surface of the top portion 103 of the tank.

In some embodiments, when the outside of the connecting member 204 is surrounded by the envelope 501 and the water-blocking disk 601 is provided on the top portion of the envelope 501, the bottom portion of the enclosure 701 may be provided with the position-limiting member 702 (not shown in FIG. 8).

By setting the enclosure, it can effectively prevent splashing liquids or water vapor from entering into an interior of the envelope and the interior of the connecting member when the liquids inside tank are stirred, henceforth avoiding contaminating the interior of a bioreactor.

The features and performances disclosed by embodiments of the present disclosure are described in further detail below in conjunction with examples.

### Test Example 1

A stirring device in t Test Example 1 includes a rotating shaft, a magnetic member, a connecting member, and an envelope, and the envelope includes a water-blocking disk with no holes, and an enclosure is disposed on an outer side of the envelope. The height of the enclosure (e.g., h is the height of the enclosure in FIG. 7A) is 5 mm, the gap between the enclosure and the envelope (e.g., d1 is the gap between the enclosure and the envelope in FIG. 7A) is 0.5 mm, the height between the envelope and a top portion of the tank (e.g., d3 is a height between the upper surface of the envelope and a lower surface of the top portion of the tank in FIG. 7A) is 0.5 mm, and the rotating shaft rotates at a speed of 80 rpm.

### Test Example 2

A stirring device in Test Example 2 includes a rotating shaft, a magnetic member, a connecting member, and an envelope, the envelope includes a water-blocking disk with no holes, and an enclosure is provided on an outer side of the envelope. The height of the enclosure is 5 mm, the gap between the enclosure and the envelope is 0.7 mm, the height between the envelope and a top portion of the tank is 0.5 mm, and the rotating shaft rotates at a speed of 80 rpm.

### Test Example 3

A stirring device in Test Example 3 includes a rotating shaft, a magnetic member, a connecting member, and an envelope, the envelope includes a water-blocking disk with no holes, and the envelope is not provided with an enclosure on an outer side of the envelope. The height between the envelope and a top portion of the tank is 0.5 mm, and the rotating shaft rotates at a speed of 80 rpm.

### Test Example 4

A stirring device in Test Example 4 includes a rotating shaft, a magnetic member, a connecting member, and an envelope, the envelope includes a water-blocking disk with a hole, and an enclosure is not disposed on an outer side of the envelope. The height between the envelope and a top portion of the tank is 1.5 mm, and the rotating shaft rotates at a speed of 50 rpm.

### Test Example 5

A stirring device in Test Example 5 includes a rotating shaft, a magnetic member, a connecting member, and an envelope, and the envelope does not include a water-blocking disk, and an enclosure is not disposed on an outer side of the envelope. The height between the envelope and a top portion of the tank is 1.5 mm, and the rotating shaft rotates at a speed of 50 rpm.

According to Test Examples 1 to 5 above, the effect of preventing condensate from entering an interior space of the envelope (also referred to as the water-blocking effect) was tested at a reactant liquid temperature of 45° C. The following test results were obtained:

| | Test time | Test results (Accumulated |
|---|---|---|
| | (Unit: h) | amount of water in mL) |
| Test Example 1 | 406 | ≤0.5 |
| Test Example 2 | 406 | ≤0.5 |
| Test Example 3 | 168 | >1 |
| Test Example 4 | 72 | >1 |
| Test Example 5 | 48 | >1 |

According to Test Examples 1-5, the effect of preventing condensate from entering the interior space of the envelope was tested at a reactant liquid temperature of 37° C., and the following test results were obtained:

| | Test time | Test results (Accumulated |
|---|---|---|
| | (Unit: h) | amount of water in mL) |
| Test Example 1 | 255.5 | ≤0.3 |
| Test Example 2 | 255.5 | ≤0.3 |
| Test Example 3 | 200 | >1 |
| Test Example 4 | 96 | >1 |
| Test Example 5 | 72 | >1 |

From the above tables, it can be seen that when the reactant liquid temperature was 45° C., an accumulated amount of water inside the envelope did not reach 0.5 mL in Test Example 1 at a test time of 406 h, and an accumulated amount of water inside the envelope did not reach 0.5 mL in Test Example 2 at a test time of 406 h; when the reactant liquid temperature was 37° C., an accumulated amount of water inside the envelope did not reach 0.3 mL at a test time of 255.5 h in Test Example 1, and an accumulated amount of water inside the envelope did not reach 0.3 mL at a test time of 255.5 h in Test Example 2. It can be seen that the gap between the enclosure and the envelope has a small effect on the water-blocking effect. The effect of preventing condensate from entering the interior space of the envelope is approximately the same when the gap between the enclosure and the envelope is set to different values.

From the above tables, it can be seen that when the reactant liquid temperature was 45° C., the accumulated amount of water inside the envelope reached 1 mL at a test time of 168 h in Test Example 3, and the accumulated amount of water inside the envelope reached 1 mL at a test time of 72 h in Test Example 4; and when the reactant liquid temperature was 37° C., the accumulated amount of water inside the envelope reached 1 mL at a test time of 200 h in Test Example 3, and an accumulated amount of water inside the envelope reached 1 mL at a test time of 96 h in Test Example 4. With only a water-blocking disk not having holes and no enclosure, the effect of preventing condensate from entering the interior space of the envelope was fair. With only a water-blocking disk having holes and no enclosure, the effect of preventing condensate from entering the inner space of the envelope was poor.

From the above tables, it can be seen that when the reactant liquid temperature was 45° C., the accumulated amount of water inside the envelope at a test time of 48 h reached 1 mL in Test Example 5; and when the reactant liquid temperature was 37° C., the accumulated amount of water inside the envelope at a test time of 72 h reached 1 mL in Test Example 5. It can be seen that the effect of preventing condensate from entering the interior space of the envelope was the worst when the water-blocking disk and the enclosure were not set up.

Through the above test, it is verified that the setting of the enclosure and the water-blocking disk can effectively prevent the condensate from entering into the interior space of the envelope, which avoids contamination of an interior space of a bioreactor when the condensate fills up the interior space of the envelope.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations thereof, are not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the count of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Therefore, by way of example, but not of limitation, alternative configurations of the embodiments of the application may be utilized in accordance with the teachings herein. Accordingly, embodiments of the present application are not limited to that precisely as shown and described.

What is claimed is:

1. A stirring device for a bioreactor, the bioreactor including a tank for accommodating a mixture of cells and liquid, wherein
the stirring device includes a rotating shaft, a magnetic member, and a connecting member,
a bottom portion of the rotating shaft is connected with the magnetic member, the magnetic member is in no contact with a bottom portion of the tank to drive the rotating shaft to rotate; the rotating shaft is in no contact with the bottom portion of the tank; and the rotating shaft is connected with a top portion of the tank through the connecting member.

2. The stirring device of claim 1, wherein the connecting member includes a bearing assembly, the bearing assembly is connected with the tank through a fixing member; the fixing member is an integral part of the tank, the fixing member is disposed on a lower surface of the top portion of the tank, and the fixing member is in no contact with the rotating shaft.

3. The stirring device of claim 1, wherein the connecting member includes a first position-limiting member disposed at the top portion of the tank and a second position-limiting member disposed at a top portion of the rotating shaft, and the first position-limiting member is connected with the second position-limiting member.

4. The stirring device of claim 1, wherein the stirring device further includes an envelope, the envelope is disposed around the connecting member, and a lower end of the envelope is fixedly connected with the rotating shaft with no gap, and an upper end of the envelope is connected with the connecting member.

5. The stirring device of claim 4, wherein the connecting member is connected with an inner side of the upper end of the envelope, and the rotating shaft is inserted through a through-hole at the lower end of the envelope, and an outer peripheral side of the rotating shaft is connected with the through-hole at the lower end of the envelope with no gap.

6. The stirring device of claim 4, wherein a water-blocking disk is disposed at a top portion of the envelope, the water-blocking disk is located above the envelope, and a bottom portion of the water-blocking disk is connected to the top portion of the envelope.

7. The stirring device of claim 6, wherein there are no holes on a disk surface of the water-blocking disk.

8. The stirring device of claim 6, wherein a center region of the water-blocking disk is higher than a rim region of the water-blocking disk.

9. The stirring device of claim 6, wherein a disk surface of the water-blocking disk is provided with a concentric circular groove, and a surface of the concentric circular groove is lower than two rim regions of the water-blocking disk.

10. The stirring device of claim 6, wherein a lower surface of the top portion of the tank includes one or more annular convex structures, the one or more annular convex structures have different diameters and co-circular; an upper surface of the water-blocking disk includes a convex loop, the convex loop includes one or more concentric rings of different diameters; and each of the one or more concentric rings of the convex loop is embedded in a gap between two adjacent annular convex structures of the one or more annular convex structures.

11. The stirring device of claim 10, wherein the convex loop is in no contact with the one or more annular convex structures along a radial direction of the tank to form a zigzag channel between the convex loop and the one or more annular convex structures.

12. The stirring device of claim 4, wherein a vacuuming and liquid-collecting member is provided inside the envelope, the vacuuming and liquid-collecting member being fixed at a bottom region inside the envelope.

13. The stirring device of claim 12, wherein at least one opening is provided in the vacuuming and liquid-collecting member.

14. The stirring device of claim 12, wherein a separating plate is disposed at a bottom portion inside the envelope, the separating plate is configured to separate the vacuuming and liquid-collecting member from a top portion of the rotating shaft; and the vacuuming and liquid-collecting member is disposed above the separating plate, and the top portion of the rotating shaft is located below the separating plate.

15. The stirring device of claim 1, wherein the tank includes an enclosure disposed underneath the top portion of the tank, the enclosure is connected with a lower surface of the top portion of the tank, at least a portion of the enclosure is disposed around the connecting member, and a height of the enclosure is greater than a distance between an upper surface of the connecting member and the lower surface of the top portion of the tank.

16. The stirring device of claim 4, wherein the tank includes an enclosure disposed underneath the top portion of the tank, the enclosure is connected with a lower surface of the top portion of the tank, at least a portion of the enclosure is disposed around the envelope, and a height of the enclosure is greater than a distance between an upper surface of the envelope and the lower surface of the top portion of the tank.

17. The stirring device of claim 15, wherein a position-limiting member is disposed at a bottom portion of the enclosure, and a radial distance between the position-limiting member and the rotating shaft along a radial direction of the tank is less than a radial width of the connecting member along the radial direction of the tank.

18. The stirring device of claim 17, wherein the position-limiting member includes one or more locking ribs distributed in an array with a center of a circle of the enclosure.

19. The stirring device of claim 2, wherein the fixing member is connected with the bearing assembly through a telescoping member.

20. A bioreactor, comprising a tank for accommodating a mixture of cells and liquid and a stirring device,
wherein the stirring device includes a rotating shaft, a magnetic member, and a connecting member,
a bottom portion of the rotating shaft is connected with the magnetic member, the magnetic member is in no contact with a bottom portion of the tank to drive the rotating shaft to rotate; the rotating shaft is in no contact with the bottom portion of the tank; and the rotating shaft is connected with a top portion of the tank through the connecting member.
